(19) United States
(12) Patent Application Publication
(10) Pub. No.: US 2024/0299923 A1
(43) Pub. Date: Sep. 12, 2024

(54) HYDROGENATION CATALYST, PREPARATION METHOD THEREFOR AND USE THEREOF

(71) Applicant: INSTITUTE OF PROCESS ENGINEERING, CHINESE ACADEMY OF SCIENCES, Beijing (CN)

(72) Inventors: Liguo WANG, Beijing (CN); Huanhuan YANG, Beijing (CN); Huiquan LI, Beijing (CN); Shuang XU, Beijing (CN); Yan CAO, Beijing (CN)

(73) Assignee: INSTITUTE OF PROCESS ENGINEERING, CHINESE ACADEMY OF SCIENCES, Beijing (CN)

(21) Appl. No.: 18/548,448

(22) PCT Filed: Apr. 9, 2021
(86) PCT No.: PCT/CN2021/086087

§ 371 Date: Nov. 20, 2023

(30) Foreign Application Priority Data
Mar. 1, 2021 (CN) 202110227355.4
Mar. 12, 2021 (CN) 202110269163.X

(51) Int. Cl. B01J 37/02; B01J 23/89; B01J 27/24; B01J 37/00; B01J 37/18; B01J 37/34; C07C 209/36

(52) U.S. Cl. CPC B01J 37/0213; B01J 23/8906; B01J 23/8913; B01J 23/892; B01J 23/8926; B01J 27/24; B01J 37/009; B01J 37/0236; B01J 37/18; B01J 37/343; C07C 209/36

(57) ABSTRACT

Disclosed are a hydrogenation catalyst, a preparation method therefor and use thereof. The hydrogenation catalyst includes a carrier and an active component supported on the carrier, wherein the carrier is nitrogen-doped carbon, and the active component is a bimetal selected from Ru—Fe, Ru—Co, Ru—Ni or Ru—Cu.

### TECHNICAL FIELD

The present application belongs to the technical field of catalysts, and relates to a hydrogenation catalyst a preparation method therefor and use thereof, for example, to a mild and efficient catalyst for hydrogenation of an aromatic nitro compound to prepare an aromatic amino compound, and a preparation method therefor and use thereof.

### BACKGROUND

Aromatic amino compounds, as an important organic intermediate, have been widely used in dyes, agricultural chemicals, pharmaceuticals, insecticides, pesticides, herbicides, plastic additives, resin synthesis, polyurethane synthesis, etc. At present, aromatic amino compounds are mainly prepared by hydrogenation of the corresponding aromatic nitro compounds. However, the commercial catalysts such as Pd/C and Pt/C need to be improved in their product selectivity in the hydrogenation reaction of aromatic nitro compounds. Thereby, it has been the focus to develop new catalysts to achieve the mild and efficient conversion of nitrobenzene to phenylamine all around the world.

Based on the literature and patent publications, the catalysts for multiphase hydrogenation of aromatic nitro compounds are mainly divided into two categories. One is precious metal catalysts, such as Pt (CN109876801A; Vilé, Gianvito, Almora-Barrios N, López, Núria, et al. Structure and Reactivity of Supported Hybrid Platinum Nanoparticles for the Flow Hydrogenation of Functionalized Nitroaromatics. Acs Catalysis, 2015, 5(6), 3767-3778.), and Pd (CN109331818 A; Gang Chen, Xun Zhu, Rong Chen, et al. Hierarchical Pd@Ni catalyst with a snow-like nanostructure on Ni foam for nitrobenzene hydrogenation. Applied Catalysis A: General, 2019,575,238-245.) etc.; these precious metal catalysts can realize conversion of aromatic nitro compounds into aromatic amino compounds through hydrogenation under mild or even normal-temperature and normal-pressure conditions, but high cost of the catalysts greatly limits the practical application of the catalysts. The other category is non-precious metal catalysts; disclosed by CN111085241A, a loaded Co-based catalyst is prepared for nitrobenzene hydrogenation, the reaction is performed at 80-150° C., the selectivity of phenylamine is 92-99%; disclosed by the paper (Hongbo Yu, Weiqiang Tang, et al. Enhanced Catalytic Performance for Hydrogenation of Substituted Nitroaromatics over Ir-Based Bimetallic Nanocatalysts. ACS applied materials & interfaces, 2019, 11, 6958-6969.), Ir-doped IrFe, IrCo and IrNi catalysts are prepared for the selective hydrogenation of aromatic nitro compounds; although the conversion rate is high, there is still some room for selectivity improvement, and the catalytic effect of non-precious metal catalysts at normal temperature and pressure needs to be improved.

In summary, for hydrogenation of aromatic nitro compounds to prepare aromatic amino compounds, the catalyst system disclosed in the prior art has severe by-product reactions and in turn has much progress to be made to increase the selectivity of aromatic amino compounds, the precious metal catalysts have high cost, and the non-precious metal catalysts have poor selectivity and require harsh reaction conditions; hence, it has important guiding significance and practical value for the production of the aromatic amino compounds to develop a mild and efficient catalyst for the hydrogenation of aromatic nitro compounds to prepare an aromatic amino compounds.

### SUMMARY

The present application is to provide a hydrogenation catalyst and a preparation method therefor and use thereof, in particular to provide a mild and efficient catalyst for hydrogenation of an aromatic nitro compound to prepare an aromatic amino compound, and a preparation method therefor and use thereof.

To achieve the object, the present application adopts the technical solutions below.

In a first aspect, the present application provides a hydrogenation catalyst, and the hydrogenation catalyst includes a carrier and an active component loaded on the carrier; the carrier is nitrogen-doped carbon, and the active component is bimetal selected from Ru—Fe, Ru—Co, Ru—Ni or Ru—Cu.

The catalyst of the present application uses nitrogen-doped carbon as a carrier and uses bimetal of Ru—Fe, Ru—Co, Ru—Ni or Ru—Cu as an active component. Compared to the prior art, the catalyst can be used in the reaction of selective hydrogenation of an aromatic nitro compound for aromatic amino production, without introducing any auxiliary agents, and realize high-activity and high-selectivity hydrogenation conversion of an aromatic nitro compound into an aromatic amino compound under mild or even normal-temperature and normal-pressure conditions

Optionally, each metal of the active component has a mass percentage of 0.01-40% in the catalyst, such as 0.01%, 0.05%, 0.1%, 1%, 3%, 5%, 8%, 10%, 15%, 20%, 25%, 30%, 35% or 40%, and any other specific point values within the numerical range can be selected and will not be enumerated exhaustively herein, and optionally, the mass percentage is 0.01-8%.

Optionally, metal ruthenium of the active component has a mass percentage of 0.01-8% in the catalyst, such as 0.01%, 0.1%, 1%, 2%, 3%, 4%, 5%, 6%, 7% or 8%, etc.

The mass percentage of the metal in the catalyst refers to the theoretical mass percentage, that is, assuming that the metal raw materials have all been fully loaded in the catalyst.

Optionally, the nitrogen-doped carbon carrier may be carbon nitride, and the carbon nitride is prepared by calcination of any one or a combination of at least two of cyanamide, dicyandiamide, tripolycyanamide, thiourea, urea or guanidine hydrochloride.

The combination of at least two includes, for example, a combination of cyanamide and dicyandiamide, a combination of dicyandiamide and tripolycyanamide, and a combination of thiourea and urea, and any other combinations can be selected and will not be enumerated exhaustively herein.

Optionally, the calcination is performed at 450-650° C., such as 450° C., 500° C., 550° C., 600° C. or 650° C., etc.; the calcination is performed for 0.5-5 h, such as 0.5 h, 1 h, 2 h, 3 h, 4 h or 5 h, etc.; any other specific point values within the above numerical ranges can be selected and will not be enumerated exhaustively herein; the calcination is performed in an atmosphere of air or an inert gas, optionally nitrogen.

Optionally, the nitrogen-doped carbon is prepared by using a polymerized ionic liquid as a precursor and using carbon nitride as a sacrificial template.

Optionally, a mass ratio of the carbon nitride to the polymerized ionic liquid is (0.2-12):1, such as 0.2:1, 1:1, 2:1, 3:1, 4:1, 5:1, 6:1, 7:1, 8:1, 9:1, 10:1, 11:1 or 12:1, etc.; any other specific point values within the numerical range can be selected and will not be enumerated exhaustively herein.

Optionally, the preparation includes mixing the polymerized ionic liquid and the carbon nitride and then calcining.

Optionally, the calcination is performed at 600-1000° C., such as 600° C., 650° C., 700° C., 750° C., 800° C., 850° C., 900° C., 950° C. or 1000° C., etc.; the calcination is performed for 0.5-5 h, such as 0.5 h, 1 h, 2 h, 3 h, 4 h or 5 h, etc.; any other specific point values within the above numerical ranges can be selected and will not be enumerated exhaustively herein; the calcination is performed in an atmosphere of an inert gas.

Optionally, the polymerized ionic liquid includes any one of compounds represented by Formula (I) to Formula (VII):

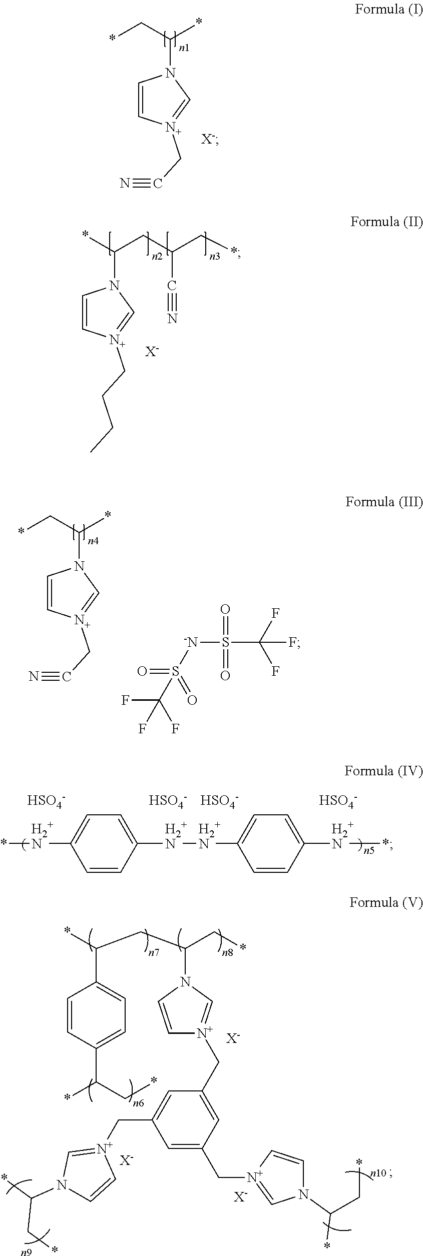

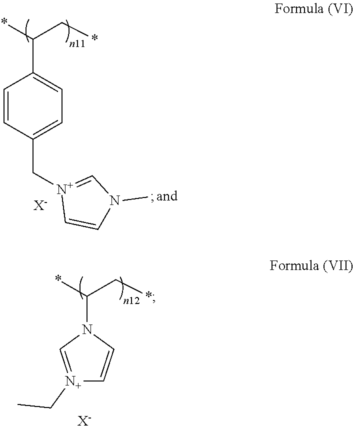

wherein, X is selected from F, Cl or Br; n1-n12 are each independently selected from integers of 4 to 1000 (such as 4, 8, 10, 15, 20, 25, 30, 50, 80, 100, 300, 500, 800 or 1000, etc.); and * denotes an extending direction for a structural unit.

In a second aspect, the present application provides a preparation method of the hydrogenation catalyst as described above, and the preparation method includes the following steps:
mixing a mixed metal precursor solution containing bimetal of Ru—Fe, Ru—Co, Ru—Ni or Ru—Cu with a nitrogen-doped carbon suspension, and performing impregnation; filtering a suspension obtained after the impregnation, and drying a filtered solid; and then performing reduction activation to obtain the catalyst.

In the present application, the preparation method for the mild and efficient catalyst for hydrogenation of an aromatic nitro compound to prepare an aromatic amino compound is facile in process and easy in industrialization.

Optionally, a preparation method for the mixed metal precursor solution containing bimetal of Ru—Fe, Ru—Co, Ru—Ni or Ru—Cu includes: mixing any one of a metal iron precursor, a metal cobalt precursor, a metal nickel precursor or a metal copper precursor with a metal ruthenium precursor and a solvent to obtain the mixed metal precursor solution.

Optionally, the solvent includes deionized water, ethanol, methanol, isopropanol, tetrahydrofuran, and other commonly used solvents.

Optionally, the metal precursor is a metal salt.

Optionally, the metal ruthenium precursor includes ruthenium trichloride and/or ruthenium acetate.

Optionally, the metal iron precursor includes ferric chloride and/or ferric nitrate and/or ferric sulfate.

Optionally, the metal cobalt precursor includes any one or a combination of at least two of cobalt chloride, cobalt nitrate, cobalt sulfate or cobalt acetate.

Optionally, the metal nickel precursor includes any one or a combination of at least two of nickel chloride, nickel nitrate or nickel sulfate.

Optionally, the metal copper precursor includes any one or a combination of at least two of copper chloride, copper nitrate or copper sulfate.

Optionally, the mixed metal precursor solution has a concentration of 0.001-0.2 g/mL, such as 0.001 g/mL, 0.005 g/mL, 0.01 g/mL, 0.05 g/mL, 0.1 g/mL, 0.15 g/mL or 0.2 g/mL, etc.; any other specific point values within the numerical range can be selected and will not be enumerated exhaustively herein.

Optionally, the nitrogen-doped carbon suspension is obtained by mixing and dispersing nitrogen-doped carbon with a solvent, wherein the solvent includes deionized water, ethanol, methanol, tetrahydrofuran, and other commonly used solvents.

Optionally, the nitrogen-doped carbon suspension has a solid-liquid ratio of 1:(10-80) g/mL, such as 1:10 g/mL, 1:20 g/mL, 1:30 g/mL, 1:40 g/mL, 1:50 g/mL, 1:60 g/mL, 1:70 g/mL or 1:80 g/mL, etc.; any other specific point values within the numerical range can be selected and will not be enumerated exhaustively herein.

Optionally, the dispersion is performed in a manner of ultrasonic dispersion, and the dispersion is performed for 0.5-12 h, such as 0.5 h, 1 h, 2 h, 4 h, 6 h, 8 h, 10 h, 11 h or 12 h, etc.; any other specific point values within the numerical range can be selected and will not be enumerated exhaustively herein.

Optionally, the impregnation is performed in a manner of stirring, and the impregnation is performed for 6-24 h, such as 6 h, 8 h, 10 h, 12 h, 15 h, 18 h, 20 h, 22 h or 24 h, etc.; any other specific point values within the numerical range can be selected and will not be enumerated exhaustively herein.

Optionally, the drying is performed at 80-120° C., such as 80° C., 90° C., 100° C., 110° C. or 120° C., etc.; the drying is performed for 6-12 h, such as 6 h, 7 h, 8 h, 9 h, 10 h, 11 h, 12 h, etc.; any other specific point values within the above numerical ranges can be selected and will not be enumerated exhaustively herein.

Optionally, the reduction activation is performed in a hydrogen atmosphere.

Optionally, the reduction activation is performed at 200-700° C., such as 200° C., 300° C., 400° C., 500° C., 600° C. or 700° C., etc.; the reduction activation is performed for 0.5-5 h, such as 0.5 h, 1 h, 2 h, 3 h, 4 h or 5 h, etc.; any other specific point values within the above numerical ranges can be selected and will not be enumerated exhaustively herein.

As an optional technical solution of the present application, the preparation method for the mild and efficient catalyst for hydrogenation of an aromatic nitro compound to prepare an aromatic amino compound includes the following steps:
(1) any one or a combination of at least two of cyanamide, dicyandiamide, tripolycyanamide, thiourea, urea or guanidine hydrochloride is calcined at 450-650° C. for 0.5-5 h in air or an inert gas, so as to obtain carbon nitride;
(2) the carbon nitride is mixed with a polymerized ionic liquid according to a mass ratio of (0.2-12):1, and calcined at 550-1000° C. for 0.5-5 h in an inert gas, so as to obtain nitrogen-doped carbon;
(3) a metal ruthenium precursor is mixed with any one of a metal iron precursor, a cobalt precursor, a nickel precursor or a copper precursor as well as a solvent, so as to obtain a mixed metal precursor solution with a precursor concentration of 0.001-0.2 g/mL; the nitrogen-doped carbon is mixed with a solvent and subjected to ultrasonic dispersion for 0.5-12 h, so as to obtain a nitrogen-doped carbon suspension with a solid-liquid ratio of 1:(10-80) g/mL;
(4) the mixed metal precursor solution and the nitrogen-doped carbon suspension obtained in step (3) are mixed and subjected to impregnation with stirring for 6-24 h;
(5) a suspension obtained after the impregnation is filtered, and a solid obtained after the filtered is dried at 80-120° C. for 6-12 h; and
(6) a solid obtained after drying is subjected to reduction activation at 200-700° C. for 0.5-6 h in a hydrogen atmosphere, so as to obtain the catalyst.

In a third aspect, the present application provides use of the hydrogenation catalyst as described above in hydrogenation of an aromatic nitro compound to prepare an aromatic amino compound.

Optionally, a method for preparing the aromatic amino compound by hydrogenation of the aromatic nitro compound includes the following steps:
subjecting an aromatic nitro compound, as a raw material, and the hydrogenation catalyst as described in the first aspect, as a catalyst, to a reaction in a hydrogen atmosphere to obtain the aromatic amino compound.

Optionally, the aromatic nitro compound includes any one of compounds represented by Formula (VIII) to Formula (XVI):

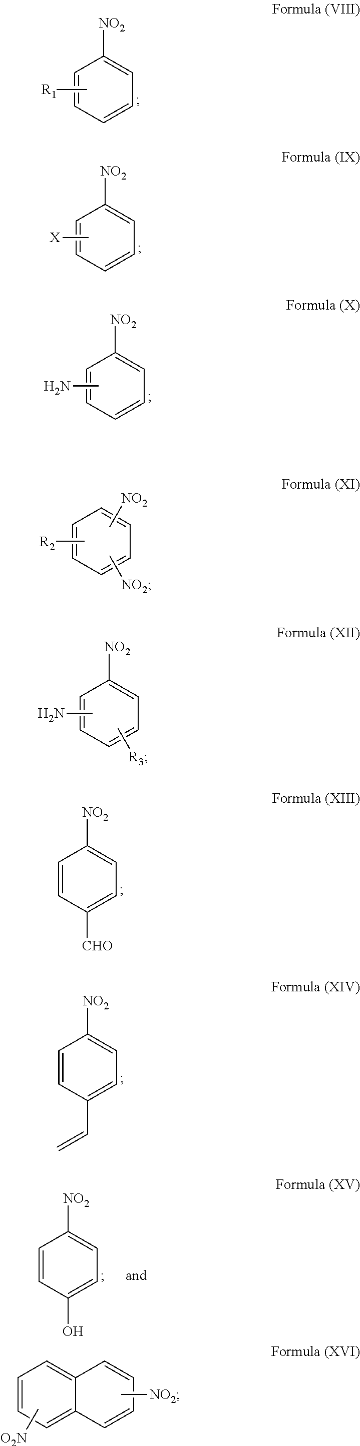

wherein, R₁, R₂, and R₃ are independently selected from H or C1-C4 alkyl; and X is selected from F, Cl or Br.

Optionally, the reaction is performed in a solvent medium, and the solvent includes any one or a combination of at least two of tetrahydrofuran, methanol, isopropanol, ethanol, propanol, cyclohexane, cyclohexylamine, n-butanol, toluene, N-methylpyrrolidone, dimethylformamide, dimethylsulfoxide or tert-butanol.

The combination of at least two includes, for example, a combination of tetrahydrofuran and methanol, a combination of isopropanol and ethanol, and a combination of cyclohexane and toluene, and any other combinations can be selected and will not be enumerated exhaustively herein.

Optionally, a usage amount of the catalyst is 0.1-30% by mass relative to the aromatic nitro compound, such as 0.1%, 1%, 2%, 5%, 10%, 15%, 20%, 25% or 30%, etc.; any specific point values within the numerical range can be selected and will not be enumerated exhaustively herein.

Optionally, the reaction is performed at −15° C. to 90° C., such as −15° C., −10° C., 0° C., 5° C., 10° C., 15° C., 20° C., 30° C., 50° C., 80° C. or 90° C., etc.; the reaction is performed for 0.1-60 h, such as 0.1 h, 0.5 h, 1 h, 5 h, 10 h, 24 h, 36 h, 48 h or 60 h, etc.; an initial pressure is 0.1-5 MPa, such as 0.1 MPa, 0.2 MPa, 0.5 MPa, 1 MPa, 2 MPa, 3 MPa, 4 MPa or 5 Mpa, etc.; any other specific point values within the above numerical ranges can be selected and will not be enumerated exhaustively herein.

As an optional technical solution of the present application, the method for preparing the aromatic amino compound by hydrogenation of the aromatic nitro compound specifically includes the following steps:

the reaction is performed in a hydrogen atmosphere by using an aromatic nitro compound as a raw material and using the catalyst as described above as a catalyst to obtain an aromatic amino compound; a reaction medium is any one or a combination of at least two of tetrahydrofuran, methanol, isopropanol, ethanol, propanol, cyclohexane, cyclohexylamine, n-butanol, toluene, N-methylpyrrolidone, dimethylformamide, dimethylsulfoxide or tert-butanol; a usage amount of the catalyst is 0.1-30% by mass relative to the aromatic nitro compound; and the reaction is performed at −15° C. to 90° C. for 0.1-60 h with an initial pressure of 0.1-5 Mpa.

Compared with the prior art, the present application has the following beneficial effects.

The catalyst of the present application uses carbon nitride, or nitrogen-doped carbon which is prepared by using carbon nitride as a sacrificial template and a polymerized ionic liquid as a carbon-nitrogen source, as a carrier, and uses bimetal of Ru—Fe, Ru—Co, Ru—Ni or Ru—Cu as an active component. On one hand, using the carbon nitride as a sacrificial template can greatly increase the specific surface area of the polymerized ionic liquid, which is conducive to the dispersion of active metals. On the other hand, the nitrogen-doped carbon carrier prepared with the polymerized ionic liquid as a carbon-nitrogen source has a high nitrogen content (more than 20 wt. %), which not only increases the basic sites of nitrogen contained in the nitrogen-doped carbon and inhibits the aromatic amino compound from further hydrogenation and condensation by-product reaction, but also provides abundant metal-N coordination opportunities for active metals, facilitating the high dispersion of metal species. Moreover, the addition of Fe, Co, Ni or Cu greatly reduces the cost of the catalyst. Therefore, the catalyst realizes the high conversion rate of hydrogenation of the aromatic nitro compound and the high selectivity of the aromatic amino compound under mild or even normal-temperature and normal-pressure conditions.

Compared with the prior art, the catalyst has low cost and can be used in the hydrogenation reaction of an aromatic nitro compound to prepare an aromatic amino compound and realize high-activity and high-selectivity conversion of an aromatic nitro compound into an aromatic amino compound under very mild conditions.

### BRIEF DESCRIPTION OF DRAWINGS

FIG. 1 is a transmission electron microscope (TEM) image of a catalyst prepared in Example 4;

FIG. 2 is a gas chromatogram in Application Example 1;

FIG. 3 is a gas chromatogram in Application Example 3;

FIG. 4 is a gas chromatogram in Application Example 6.

### DETAILED DESCRIPTION

The technical solutions of the present application are further explained below in terms of the specific embodiments. It should be apparent to those skilled in the art that the examples are merely used for a better understanding of the present application and should not be regarded as a specific limitation of the present application.

Conditions for the gas chromatography analysis involved in the application examples below include: chromatographic column: RTX-5; a column temperature was initially maintained at 80° C. for 1 min, and then it increased to 125° C. at 10° C./min and was maintained for 2 min. The temperature eventually increased to 230° C. at 20° C./min and was maintained for 7.25 min; a control mode is pressure control, a pressure is 50 kPa, a purge flow is 3 mL/min and a split ratio is 30; a detection temperature is 250° C.

### Example 1

This example provides a mild and efficient catalyst for hydrogenation of an aromatic nitro compound to prepare an aromatic amino compound, and a preparation method for the catalyst is as follows:
(1) urea was put in a crucible, covered with a lid, and calcined in a muffle furnace at 550° C. for 4 h, and a solid obtained was washed with deionized water and ethanol for three times individually, and then dried in a blast drying oven at 100° C. for 12 h to obtain carbon nitride;
(2) the carbon nitride was mixed with a polymerized ionic liquid having the following structure (with a number average molecular mass of 100000) according to a mass ratio of 2:1, and calcined in a tubular furnace at 650° C. for 1 h in a nitrogen atmosphere to obtain nitrogen-doped carbon (for a preparation method for the polymerized ionic liquid, see Reference: Su-Yun Zhang, Qiang Zhang, Miao Zhang et al., Poly(ionic liquid) composites, Chemical Society Reviews, 2020, 49, 1726);

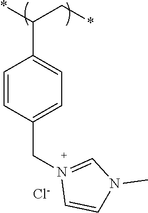

(3) 0.10 g of RuCl₃ and 0.5 g of FeCl₃·6H₂O were dissolved in 10 mL of deionized water to obtain a mixed metal precursor solution with a concentration of 0.019 g/mL; 1.74 g of the nitrogen-doped carbon powder obtained in step (2) was dispersed in 60 mL of deionized water and subjected to ultrasonic treatment for 30 min to obtain a nitrogen-doped carbon suspension;

(4) the mixed metal precursor solution obtained in step (3) and the nitrogen-doped carbon suspension were mixed according to a volume ratio of 1:6 and subjected to impregnation with stirring for 12 h;
(5) a suspension obtained after the impregnation was filtered, and a solid obtained after the filtered was dried at 110° C. for 8 h; and
(6) a solid obtained after drying was put in a tube furnace and subjected to reduction activation at 300° C. for 4 h in a hydrogen atmosphere to obtain the catalyst.

### Example 2

This example provides a mild and efficient catalyst for hydrogenation of an aromatic nitro compound to prepare an aromatic amino compound, and a preparation method for the catalyst is as follows:
(1) urea and tripolycyanamide were mixed in a crucible according to a mass ratio of 3:1, covered with a lid, and calcined in a muffle furnace at 600° C. for 3 h, and a solid obtained was washed with deionized water and ethanol for three times individually, and then dried in a blast drying oven at 100° C. for 12 h to obtain carbon nitride;
(2) the carbon nitride was mixed with a polymerized ionic liquid having the following structure (with a number average molecular mass of 150000) according to a mass ratio of 3:1, and calcined in a tubular furnace at 700° C. for 1 h in a nitrogen atmosphere to obtain nitrogen-doped carbon (for a preparation method for the polymerized ionic liquid, see Reference: Su-Yun Zhang, Qiang Zhang, Miao Zhang et al., Poly(ionic liquid) composites, Chemical Society Reviews, 2020, 49, 1726);

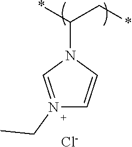

(3) 0.13 g of RuCl₃ and 0.4 g of Co(NO₃)₃·6H₂O were dissolved in 20 mL of deionized water to obtain a mixed metal precursor solution with a concentration of 0.013 g/mL; 2.00 g of the nitrogen-doped carbon powder obtained in step (2) was dispersed in 60 mL of deionized water and subjected to ultrasonic treatment for 30 min to obtain a nitrogen-doped carbon suspension;
(4) the mixed metal precursor solution obtained in step (3) and the nitrogen-doped carbon suspension were mixed according to a volume ratio of 1:3 and subjected to impregnation with stirring for 16 h;
(5) a suspension obtained after the impregnation was filtered, and a solid obtained after the filtered was dried at 100° C. for 8 h; and
(6) a solid obtained after drying was put in a tube furnace and subjected to reduction activation at 350° C. for 5 h in a hydrogen atmosphere to obtain the mild and efficient catalyst.

### Example 3

This example provides a mild and efficient catalyst for hydrogenation of an aromatic nitro compound to prepare an aromatic amino compound, and a preparation method for the catalyst is as follows:
(1) tripolycyanamide was put in a crucible, covered with a lid, and calcined in a muffle furnace at 550° C. for 4 h, and a solid obtained was washed with deionized water and ethanol for three times individually, and then dried in a blast drying oven at 100° C. for 12 h to obtain carbon nitride;
(2) the carbon nitride was mixed with a polymerized ionic liquid having the following structure (with a number average molecular mass of 80000) according to a mass ratio of 5:1, and calcined in a tubular furnace at 750° C. for 1 h in a nitrogen atmosphere to obtain nitrogen-doped carbon (for a preparation method for the polymerized ionic liquid, see Reference: Su-Yun Zhang, Qiang Zhang, Miao Zhang et al., Poly(ionic liquid) composites, Chemical Society Reviews, 2020, 49, 1726);

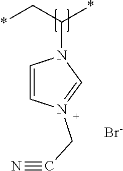

(3) 0.10 g of RuCl₃ and 0.6 g of Ni(NO₃)₃·6H₂O were dissolved in 20 mL of deionized water to obtain a mixed metal precursor solution with a concentration of 0.011 g/mL; 2.00 g of the nitrogen-doped carbon powder obtained in step (2) was dispersed in 70 mL of deionized water and subjected to ultrasonic treatment for 30 min to obtain a nitrogen-doped carbon suspension;
(4) the mixed metal precursor solution obtained in step (3) and the nitrogen-doped carbon suspension were mixed according to a volume ratio of 2:7 and subjected to impregnation with stirring for 24 h;
(5) a suspension obtained after the impregnation was filtered, and a solid obtained after the filtered was dried at 90° C. for 8 h; and
(6) a solid obtained after drying was put in a tube furnace and subjected to reduction activation at 400° C. for 3 h in a hydrogen atmosphere to obtain the catalyst.

### Example 4

This example provides a mild and efficient catalyst for hydrogenation of an aromatic nitro compound to prepare an aromatic amino compound, and a preparation method for the catalyst is as follows:
(1) dicyandiamide was put in a crucible, covered with a lid, and calcined in a muffle furnace at 450° C. for 6 h, and a solid obtained was washed with deionized water and ethanol for three times individually, and then dried in a blast drying oven at 100° C. for 12 h to obtain carbon nitride;
(2) the carbon nitride was mixed with a polymerized ionic liquid having the following structure according to a mass ratio of 10:1, and calcined in a tubular furnace at 800° C. for 0.5 h in a nitrogen atmosphere to obtain nitrogen-doped carbon (for a preparation method for the polymerized ionic liquid, see Reference: Su-Yun Zhang, Qiang Zhang, Miao Zhang et al., Poly(ionic liquid) composites, Chemical Society Reviews, 2020, 49, 1726);

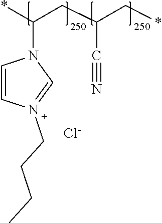

(3) 0.40 g of ruthenium acetate and 0.2 g of CuSO₄·5H₂O were dissolved in 20 mL of deionized water to obtain a mixed metal precursor solution with a concentration of 0.031 g/mL; 2.00 g of the nitrogen-doped carbon powder obtained in step (2) was dispersed in 80 mL of deionized water and subjected to ultrasonic treatment for 30 min to obtain a nitrogen-doped carbon suspension;
(4) the mixed metal precursor solution obtained in step (3) and the nitrogen-doped carbon suspension were mixed according to a volume ratio of 1:4 and subjected to impregnation with stirring for 24 h;
(5) a suspension obtained after the impregnation was filtered, and a solid obtained after the filtered was dried at 120° C. for 6 h; and
(6) a solid obtained after drying was put in a tube furnace and subjected to reduction activation at 500° C. for 1.5 h in a hydrogen atmosphere to obtain the catalyst. The morphology of the catalyst was characterized by a field-emission transmission electron microscope (FEI Tecnai G2 F30) produced by FEI Company in the United States. The TEM image is shown in FIG. 1. As can be seen from FIG. 1, the catalyst using a carrier of nitrogen-doped carbon shows a lamellar shape, and the active metal particles are well dispersed on the carrier.

### Example 5

This example provides a mild and efficient catalyst for hydrogenation of an aromatic nitro compound to prepare an aromatic amino compound, and a preparation method for the catalyst is as follows:
(1) thiourea was put in a crucible, covered with a lid, and calcined in a muffle furnace at 700° C. for 2 h, and a solid obtained was washed with deionized water and ethanol for three times individually, and then dried in a blast drying oven at 100° C. for 12 h to obtain carbon nitride;
(2) the carbon nitride was mixed with a polymerized ionic liquid having the following structure (with a number average molecular mass of 150000) according to a mass ratio of 12:1, and calcined in a tubular furnace at 750° C. for 1.5 h in a nitrogen atmosphere to obtain nitrogen-doped carbon (for a preparation method for the polymerized ionic liquid, see Reference: Su-Yun Zhang, Qiang Zhang, Miao Zhang et al., Poly(ionic liquid) composites, Chemical Society Reviews, 2020, 49, 1726);

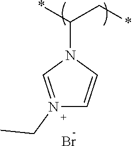

(3) 0.35 g of ruthenium acetate and 0.1 g of Co(CH₃COO)₂·4H₂O were dissolved in 20 mL of deionized water to obtain a mixed metal precursor solution with a concentration of 0.045 g/mL; 2.00 g of the nitrogen-doped carbon powder obtained in step (2) was dispersed in 80 mL of deionized water and subjected to ultrasonic treatment for 30 min to obtain a nitrogen-doped carbon suspension;
(4) the mixed metal precursor solution obtained in step (3) and the nitrogen-doped carbon suspension were mixed according to a volume ratio of 1:4 and subjected to impregnation with stirring for 18 h;
(5) a suspension obtained after the impregnation was filtered, and a solid obtained after the filtered was dried at 100° C. for 8 h; and
(6) a solid obtained after drying was put in a tube furnace and subjected to reduction activation at 400° C. for 3 h in a hydrogen atmosphere to obtain the catalyst.

### Example 6

This example provides a mild and efficient catalyst for hydrogenation of an aromatic nitro compound to prepare an aromatic amino compound, and a preparation method for the catalyst is as follows:
(1) urea was put in a crucible, covered with a lid, and calcined in a muffle furnace at 650° C. for 3 h, and a solid obtained was washed with deionized water and ethanol for three times individually, and then dried in a blast drying oven at 100° C. for 12 h to obtain carbon nitride;
(2) the carbon nitride was mixed with a polymerized ionic liquid having the following structure (with a number average molecular mass of 200000) according to a mass ratio of 10:1, and calcined in a tubular furnace at 700° C. for 0.5 h in a nitrogen atmosphere to obtain nitrogen-doped carbon (for a preparation method for the polymerized ionic liquid, see Reference: Su-Yun Zhang, Qiang Zhang, Miao Zhang et al., Poly(ionic liquid) composites, Chemical Society Reviews, 2020, 49, 1726);

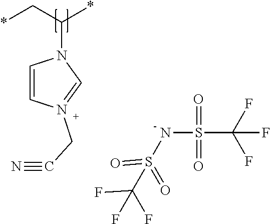

(3) 0.04 g of RuCl₃ and 0.5 g of Ni(NO₃)₂·6H₂O were dissolved in 10 mL of deionized water to obtain a mixed metal precursor solution with a concentration of 0.004 g/mL; 2.00 g of the nitrogen-doped carbon powder obtained in step (2) was dispersed in 60 mL of deionized water and subjected to ultrasonic treatment for 30 min to obtain a nitrogen-doped carbon suspension;
(4) the mixed metal precursor solution obtained in step (3) and the nitrogen-doped carbon suspension were mixed according to a volume ratio of 1:6 and subjected to impregnation with stirring for 12 h;
(5) a suspension obtained after the impregnation was filtered, and a solid obtained after the filtered was dried at 110° C. for 8 h; and
(6) a solid obtained after drying was put in a tube furnace and subjected to reduction activation at 500° C. for 4 h in a hydrogen atmosphere to obtain the catalyst.

### Example 7

This example provides a mild and efficient catalyst for hydrogenation of an aromatic nitro compound to prepare an aromatic amino compound, and a preparation method for the catalyst is as follows:
(1) thiourea was put in a crucible, covered with a lid, and calcined in a muffle furnace at 550° C. for 4 h, and a solid obtained was washed with deionized water and ethanol for three times individually, and then dried in a blast drying oven at 100° C. for 12 h to obtain carbon nitride;
(2) the carbon nitride was mixed with a polymerized ionic liquid having the following structure (with a number average molecular mass of 250000) according to a mass ratio of 12:1, and calcined in a tubular furnace at 750° C. for 3 h in a nitrogen atmosphere to obtain nitrogen-doped carbon (for a preparation method for the polymerized ionic liquid, see Reference: Ling Miao, Hui Duan, Mingxian Liu et al., Poly(ionic liquid)-derived, N, S-codoped ultramicroporous carbon nanoparticles for supercapacitors, Chemical Engineering Journal, 2017, 317, 651-659);

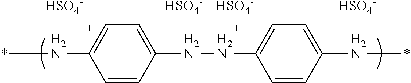

(3) 0.015 g of ruthenium acetate and 0.1 g of Ni(NO₃)₂·6H₂O were dissolved in 5 mL of deionized water to obtain a mixed metal precursor solution with a concentration of 0.003 g/mL; 2.00 g of the nitrogen-doped carbon powder obtained in step (2) was dispersed in 60 mL of deionized water and subjected to ultrasonic treatment for 30 min to obtain a nitrogen-doped carbon suspension;
(4) the mixed metal precursor solution obtained in step (3) and the nitrogen-doped carbon suspension were mixed according to a volume ratio of 1:12 and subjected to impregnation with stirring for 18 h;
(5) a suspension obtained after the impregnation was filtered, and a solid obtained after the filtered was dried at 100° C. for 8 h; and
(6) a solid obtained after drying was put in a tube furnace and subjected to reduction activation at 400° C. for 3 h in a hydrogen atmosphere to obtain the catalyst.

### Example 8

(1) Urea was put in a crucible, covered with a lid, and calcined in a muffle furnace at 550° C. for 4 h, and a solid obtained was washed with deionized water and ethanol for three times individually, and then dried in a blast drying oven at 100° C. for 12 h to obtain carbon nitride;
(2) 0.02 g of ruthenium acetate and 0.2 g of Ni(NO3)3·6H2O were dissolved in 5 mL of deionized water to obtain a mixed metal precursor solution with a concentration of 0.003 g/mL; 2.00 g of the nitrogen-doped carbon powder obtained in step (2) was dispersed in 60 mL of deionized water and subjected to ultrasonic treatment for 30 min to obtain a nitrogen-doped carbon suspension;
(3) the mixed metal precursor solution obtained in step (3) and the carbon nitride suspension were mixed according to a volume ratio of 1:12 and subjected to impregnation with stirring for 18 h;
(4) a suspension obtained after the impregnation was filtered, and a solid obtained after the filtered was dried at 100° C. for 8 h; and
(5) a solid obtained after drying was put in a tube furnace and subjected to reduction activation at 400° C. for 3 h in a hydrogen atmosphere to obtain the catalyst.

### Comparative Example 1

This comparative example provides a mild and efficient catalyst for selective hydrogenation of an aromatic nitro compound to prepare an aromatic amino compound, which uses nitrogen-doped carbon, generated by calcining a polymerized ionic liquid solely, as a carrier. A preparation method for the catalyst is as follows:
(1) a polymerized ionic liquid having the following structure was calcined in a tubular furnace at 800° C. for 0.5 h in a nitrogen atmosphere to obtain nitrogen-doped carbon;

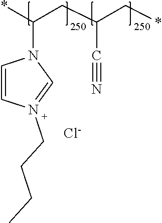

(2) 0.40 g of ruthenium acetate and 0.2 g of CuSO₄·5H₂O were dissolved in 20 mL of deionized water to obtain a mixed metal precursor solution; 2.00 g of the nitrogen-doped carbon powder obtained in step (2) was dispersed in 80 mL of deionized water and subjected to ultrasonic treatment for 30 min to obtain a nitrogen-doped carbon suspension;
(3) the mixed metal precursor solution obtained in step (2) and the nitrogen-doped carbon suspension were mixed according to a volume ratio of 1:4 and subjected to impregnation with stirring for 24 h;
(4) a suspension obtained after the impregnation was filtered, and a solid obtained after the filtered was dried at 120° C. for 6 h; and
(5) a solid obtained after drying was put in a tube furnace and subjected to reduction activation at 500° C. for 1.5 h in a hydrogen atmosphere to obtain the catalyst.

### Comparative Example 2

This comparative example provides a mild and efficient catalyst for selective hydrogenation of an aromatic nitro compound to prepare an aromatic amino compound, which uses activated carbon as a carrier. A preparation method for the catalyst is as follows:
(1) 0.10 g of RuCl3 and 0.5 g of FeCl3·6H2O were dissolved in 10 mL of deionized water to obtain a metal precursor solution; 1.74 g of activated carbon was dispersed in 60 mL of deionized water and subjected to ultrasonic treatment for 30 min to obtain a suspension;
(2) the mixed metal precursor solution obtained in step (1) and the suspension were mixed according to a volume ratio of 1:6 and subjected to impregnation with stirring for 12 h;
(3) a suspension obtained after the impregnation was filtered, and a solid obtained after the filtered was dried at 110° C. for 8 h; and
(4) a solid obtained after drying was put in a tube furnace and subjected to reduction activation at 300° C. for 4 h in a hydrogen atmosphere to obtain the catalyst.

### Comparative Example 3

This comparative example provides a mild and efficient catalyst for selective hydrogenation of an aromatic nitro compound to prepare an aromatic amino compound, which uses metal Cu solely as an active component. A preparation method for the catalyst is as follows:
(1) a polymerized ionic liquid having the following structure was calcined in a tubular furnace at 800° C. for 0.5 h in a nitrogen atmosphere to obtain nitrogen-doped carbon;

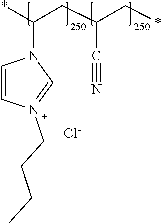

(2) 0.2 g of CuSO₄·5H₂O were dissolved in 20 mL of deionized water to obtain a metal precursor solution; 2.00 g of the nitrogen-doped carbon powder obtained in step (2) was dispersed in 80 mL of deionized water and subjected to ultrasonic treatment for 30 min to obtain a nitrogen-doped carbon suspension;
(3) the metal precursor solution obtained in step (2) and the nitrogen-doped carbon suspension were mixed according to a volume ratio of 1:4 and subjected to impregnation with stirring for 24 h;
(4) a suspension obtained after the impregnation was filtered, and a solid obtained after the filtered was dried at 120° C. for 6 h; and
(5) a solid obtained after drying was put in a tube furnace and subjected to reduction activation at 500° C. for 1.5 h in a hydrogen atmosphere to obtain the catalyst.

### Comparative Example 4

This comparative example provides a mild and efficient catalyst for selective hydrogenation of an aromatic nitro compound to prepare an aromatic amino compound, which uses metal Ni solely as an active component. A preparation method for the catalyst is as follows:
(1) a polymerized ionic liquid having the following structure was calcined in a tubular furnace at 800° C. for 0.5 h in a nitrogen atmosphere to obtain nitrogen-doped carbon;

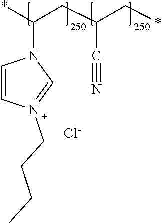

(2) 0.2 g of Ni(NO₃)₂·6H₂O were dissolved in 20 mL of deionized water to obtain a metal precursor solution; 2.00 g of the nitrogen-doped carbon powder obtained in step (2) was dispersed in 80 mL of deionized water and subjected to ultrasonic treatment for 30 min to obtain a nitrogen-doped carbon suspension;
(3) the metal precursor solution obtained in step (2) and the nitrogen-doped carbon suspension were mixed according to a volume ratio of 1:4 and subjected to impregnation with stirring for 24 h;
(4) a suspension obtained after the impregnation was filtered, and a solid obtained after the filtered was dried at 120° C. for 6 h; and
(5) a solid obtained after drying was put in a tube furnace and subjected to reduction activation at 500° C. for 1.5 h in a hydrogen atmosphere to obtain the catalyst.

### Comparative Example 5

This comparative example provides a mild and efficient catalyst for selective hydrogenation of an aromatic nitro compound to prepare an aromatic amino compound, and active components, Ru and Cu, each have a content of 50%. A preparation method for the catalyst is as follows:
(1) a polymerized ionic liquid having the following structure was calcined in a tubular furnace at 800° C. for 0.5 h in a nitrogen atmosphere to obtain nitrogen-doped carbon;

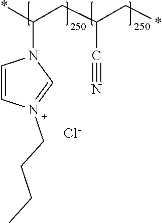

(2) 5.5 g of ruthenium acetate and 15.7 g of CuSO₄·5H₂O were dissolved in 100 mL of deionized water to obtain a metal precursor solution; 2.00 g of the nitrogen-doped carbon powder obtained in step (2) was dispersed in 80 mL of deionized water and subjected to ultrasonic treatment for 30 min to obtain a nitrogen-doped carbon suspension;
(3) the metal precursor solution obtained in step (2) and the nitrogen-doped carbon suspension were mixed according to a volume ratio of 5:4 and subjected to impregnation with stirring for 24 h;
(4) a suspension obtained after the impregnation was filtered, and a solid obtained after the filtered was dried at 120° C. for 6 h; and
(5) a solid obtained after drying was put in a tube furnace and subjected to reduction activation at 500° C. for 1.5 h in a hydrogen atmosphere to obtain the catalyst.

### Application Example 1

This application example provides a method for preparing an aromatic amino compound by using an aromatic nitro compound as a raw material, which includes the following steps:

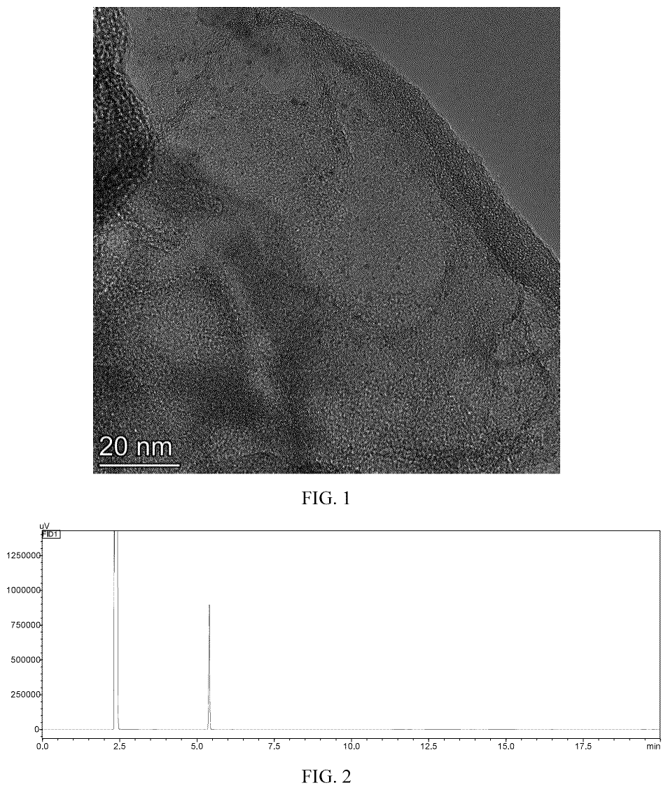

0.62 g of nitrobenzene, 0.12 g of the catalyst prepared in Example 1, and 15 mL of ethanol were added into a stainless steel autoclave, the autoclave was purged with nitrogen and hydrogen for three times individually and finally filled up with 0.1 MPa of H2; after the sealing condition was confirmed to be good, the autoclave was maintained at a normal temperature of 20° C. for 5 h; after the reaction was completed, the gas in the autoclave was released, the autoclave was opened, the catalyst was separated by centrifugation, and the supernatant was analyzed by gas chromatography, the results of which are shown in Table 1. The chromatogram of gas chromatographic is shown in FIG. 2 (the ethanol peak and tetraphenylamine peak are shown in the figure in sequence from left to right).

### Application Example 2

This application example provides a method for preparing an aromatic amino compound using an aromatic nitro compound as a raw material, operations of which differ from Application Example 1 only in that the reaction had a holding period of 3 h instead of 5 h, and other conditions are the same as those in Application Example 1. The supernatant was analyzed by gas chromatography, and the results are shown in Table 1.

### Application Example 3

This application example provides a method for preparing an aromatic amino compound using an aromatic nitro compound as a raw material, operations of which differ from Application Example 1 only in that nitrobenzene was replaced with p-dinitrobenzene, and other conditions are the same as those in Application Example 1. The supernatant was analyzed by gas chromatography, the results of which are shown in Table 1. The chromatogram of gas chromatographic is shown in FIG. 3 (the ethanol peak and p-phenylenediamine peak are shown in the figure in sequence from left to right).

### Application Example 4

This application example provides a method for preparing an aromatic amino compound using an aromatic nitro compound as a raw material, operations of which differ from Application Example 1 only in that the reaction was performed at 10° C. instead of 20° C., and other conditions are the same as those in Application Example 1. The supernatant was analyzed by gas chromatography, the results of which are shown in Table 1.

### Application Example 5

This application example provides a method for preparing an aromatic amino compound using an aromatic nitro compound as a raw material, operations of which differ from Application Example 1 only in that the reaction was performed at 90° C. instead of 20° C., and other conditions are the same as those in Application Example 1. The supernatant was analyzed by gas chromatography, the results of which are shown in Table 1.

### Application Example 6

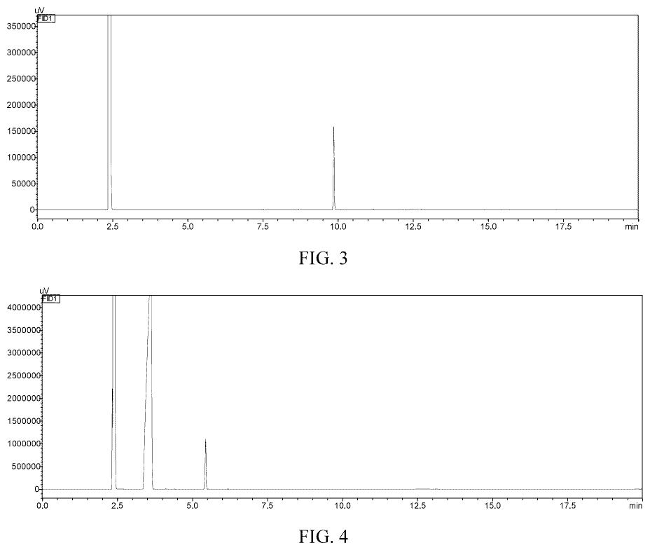

This application example provides a method for preparing an aromatic amino compound using an aromatic nitro compound as a raw material, operations of which differ from Application Example 1 only in that ethanol was replaced with N,N-dimethylformamide, and other conditions are the same as those in Application Example 1. The supernatant was analyzed by gas chromatography, the results of which are shown in Table 1. The chromatogram of gas chromatographic is shown in FIG. 4 (the ethanol peak, N,N-dimethylformamide peak and phenylamine peak are shown in the figure in sequence from left to right).

### Application Example 7

This application example provides a method for preparing an aromatic amino compound using an aromatic nitro compound as a raw material, operations of which differ from Application Example 1 only in that the catalyst prepared in Example 1 was replaced with the catalyst prepared in Example 2, and other conditions are the same as those in Application Example 1. The supernatant was analyzed by gas chromatography, the results of which are shown in Table 1.

### Application Example 8

This application example provides a method for preparing an aromatic amino compound using an aromatic nitro compound as a raw material, operations of which differ from Application Example 1 only in that the catalyst prepared in Example 1 was replaced with the catalyst prepared in Example 3, and other conditions are the same as those in Application Example 1. The supernatant was analyzed by gas chromatography, the results of which are shown in Table 1.

### Application Example 9

This application example provides a method for preparing an aromatic amino compound using an aromatic nitro compound as a raw material, operations of which differ from Application Example 1 only in that the catalyst prepared in Example 1 was replaced with the catalyst prepared in Example 4, and other conditions are the same as those in Application Example 1. The supernatant was analyzed by gas chromatography, the results of which are shown in Table 1.

### Application Example 10

This application example provides a method for preparing an aromatic amino compound using an aromatic nitro compound as a raw material, operations of which differ from Application Example 1 only in that the catalyst prepared in Example 1 was replaced with the catalyst prepared in Example 5, and other conditions are the same as those in Application Example 1. The supernatant was analyzed by gas chromatography, the results of which are shown in Table 1.

### Application Example 11

This application example provides a method for preparing an aromatic amino compound using an aromatic nitro compound as a raw material, operations of which differ from Application Example 1 only in that the catalyst prepared in Example 1 was replaced with the catalyst prepared in Example 6, and other conditions are the same as those in Application Example 1. The supernatant was analyzed by gas chromatography, the results of which are shown in Table 1.

### Application Example 12

This application example provides a method for preparing an aromatic amino compound using an aromatic nitro compound as a raw material, operations of which differ from Application Example 1 only in that the catalyst prepared in Example 1 was replaced with the catalyst prepared in Example 7, and other conditions are the same as those in Application Example 1. The supernatant was analyzed by gas chromatography, the results of which are shown in Table 1.

### Application Example 13

This application example provides a method for preparing an aromatic amino compound using an aromatic nitro compound as a raw material, operations of which differ from Application Example 1 only in that the catalyst prepared in Example 1 was replaced with the catalyst prepared in Example 8, and other conditions are the same as those in Application Example 1. The supernatant was analyzed by gas chromatography, the results of which are shown in Table 1.

### Comparative Application Example 1

This comparative application example provides a method for preparing an aromatic amino compound using an aromatic nitro compound as a raw material, operations of which differ from Application Example 1 only in that the catalyst prepared in Example 1 was replaced with the catalyst prepared in Comparative Example 1, and other conditions are the same as those in Application Example 1. The supernatant was analyzed by gas chromatography, the results of which are shown in Table 1.

### Comparative Application Example 2

This comparative application example provides a method for preparing an aromatic amino compound using an aromatic nitro compound as a raw material, operations of which differ from Application Example 1 only in that the catalyst prepared in Example 1 was replaced with the catalyst prepared in Comparative Example 2, and other conditions are the same as those in Application Example 1. The supernatant was analyzed by gas chromatography, the results of which are shown in Table 1.

### Comparative Application Example 3

This comparative application example provides a method for preparing an aromatic amino compound using an aromatic nitro compound as a raw material, operations of which differ from Application Example 1 only in that the catalyst prepared in Example 1 was replaced with the catalyst prepared in Comparative Example 3, and other conditions are the same as those in Application Example 1. The supernatant was analyzed by gas chromatography, the results of which are shown in Table 1.

### Comparative Application Example 4

This comparative application example provides a method for preparing an aromatic amino compound using an aromatic nitro compound as a raw material, operations of which differ from Application Example 1 only in that the catalyst prepared in Example 1 was replaced with the catalyst prepared in Comparative Example 4, and other conditions are the same as those in Application Example 1. The supernatant was analyzed by gas chromatography, the results of which are shown in Table 1.

### Comparative Application Example 5

This comparative application example provides a method for preparing an aromatic amino compound using an aromatic nitro compound as a raw material, operations of which differ from Application Example 1 only in that the catalyst prepared in Example 1 was replaced with the catalyst prepared in Comparative Example 5, and other conditions are the same as those in Application Example 1. The supernatant was analyzed by gas chromatography, the results of which are shown in Table 1.

TABLE 1

| | | Conversion rate of | Selectivity of |
|---|---|---|---|
| | | aromatic nitro | aromatic amino |
| | | compound | compound |
| Group | Substrate | (%) | (%) |
| Application | nitrobenzene | >99 | 98 |
| Example 1 | | | |
| Application | nitrobenzene | >99 | 98 |
| Example 2 | | | |
| Application | p- | >99 | 98 |
| Example 3 | dinitrobenzene | | |
| Application | nitrobenzene | >99 | >99 |
| Example 4 | | | |
| Application | nitrobenzene | >99 | 97 |
| Example 5 | | | |
| Application | nitrobenzene | >99 | >99 |
| Example 6 | | | |
| Application | nitrobenzene | >99 | >99 |
| Example 7 | | | |
| Application | nitrobenzene | >99 | >99 |
| Example 8 | | | |
| Application | nitrobenzene | >99 | 98 |
| Example 9 | | | |
| Application | nitrobenzene | >99 | >99 |
| Example 10 | | | |
| Application | nitrobenzene | >99 | >99 |
| Example 11 | | | |
| Application | nitrobenzene | >99 | 98 |
| Example 12 | | | |
| Application | nitrobenzene | >99 | 97 |
| Example 13 | | | |
| Comparative | nitrobenzene | 15 | 90 |
| Application | | | |
| Example 1 | | | |
| Comparative | nitrobenzene | 0 | 0 |
| Application | | | |
| Example 2 | | | |
| Comparative | nitrobenzene | 0 | 0 |
| Application | | | |
| Example 3 | | | |
| Comparative | nitrobenzene | 0 | 0 |
| Application | | | |
| Example 4 | | | |
| Comparative | nitrobenzene | 12 | 78 |
| Application | | | |
| Example 5 | | | |

The data in Table 1 show that that in a case where the bimetallic catalyst prepared by the method of the present application is used for catalyzing the hydrogenation of the aromatic nitro compound to synthesize the aromatic amino compound, the conversion rate of aromatic nitro compound is more than 99%, and the selectivity of aromatic amino compound is more than 97%. The reason is that the catalyst prepared by the present application includes a porous nitrogen-doped carbon material and bimetal loaded on the carrier; the nitrogen contained in the carrier is used as a basic site, so that the catalyst, without any auxiliary agent added, can effectively inhibit the generation of azo compounds in the hydrogenation process of aromatic nitro compound and the benzene ring hydrogenation, deamination and condensation by-product reaction in the generation process of aromatic amino compound. Moreover, the nitrogen contained in the carrier enhances the interaction between the carrier and the bimetallic species. The reaction can be carried out under relatively mild temperature and pressure, and the conversion rate of aromatic nitro compound in hydrogenation and the selectivity of aromatic amino compound are high.

The applicant states that although the process of the present application is illustrated by the above examples, the present application is not limited to the above process steps, which means that the present application is not necessarily rely on the above process steps to be implemented.

1. A hydrogenation catalyst, comprising a carrier and an active component loaded on the carrier; the carrier is nitrogen-doped carbon, and the active component is bimetal selected from Ru—Fe, Ru—Co, Ru—Ni or Ru—Cu.

2. The hydrogenation catalyst according to claim 1, wherein the nitrogen-doped carbon is carbon nitride or nitrogen-doped carbon which is prepared by using a polymerized ionic liquid as a precursor.

3. The hydrogenation catalyst according to claim 2, wherein, in a case where the polymerized ionic liquid is used as a precursor, the nitrogen-doped carbon is prepared by using carbon nitride as a sacrificial template.

4. The hydrogenation catalyst according to claim 2, wherein the carbon nitride is prepared by calcining any one or a combination of at least two of cyanamide, dicyandiamide, tripolycyanamide, thiourea, urea or guanidine hydrochloride;
optionally, the calcination is performed at 450-650° C. for 0.5-5 h, and the calcination is performed in an atmosphere of air or an inert gas, optionally nitrogen.

5. The hydrogenation catalyst according to claim 3, wherein a mass ratio of the carbon nitride to the polymerized ionic liquid is (0.2-12):1;
optionally, the preparation comprises mixing the polymerized ionic liquid and the carbon nitride and then calcining;
optionally, the calcination is performed at 550-1000° C. for 0.5-5 h, and the calcination is performed in an atmosphere of an inert gas;
optionally, the polymerized ionic liquid comprises any one of compounds represented by Formula (I) to Formula (VII): 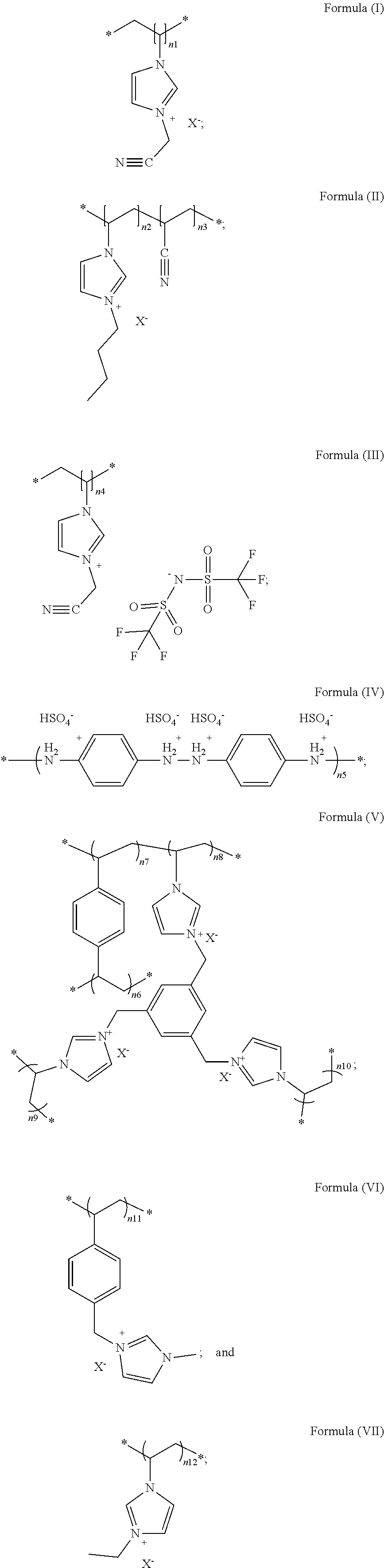
wherein X is selected from F, Cl or Br, n1-n12 are each independently selected from integers of 4 to 1000; and * denotes an extending direction for a structural unit.

6. The hydrogenation catalyst according to claim 1, wherein each metal of the active component has a mass percentage of 0.01-40% in the catalyst, optionally 0.01-8%;
optionally, metal ruthenium of the active component has a mass percentage of 0.01-8% in the catalyst.

7. A preparation method for the hydrogenation catalyst according to claim 1, comprising the following steps:
mixing a mixed metal precursor solution containing bimetal of Ru—Fe, Ru—Co, Ru—Ni or Ru—Cu with a nitrogen-doped carbon suspension, and performing impregnation; filtering a suspension obtained after the impregnation, and drying a filtered solid; and then performing reduction activation to obtain the catalyst.

8. The preparation method according to claim 7, wherein a preparation method for the mixed metal precursor solution containing bimetal of Ru—Fe, Ru—Co, Ru—Ni or Ru—Cu comprises: mixing any one of a metal iron precursor, a metal cobalt precursor, a metal nickel precursor or a metal copper precursor with a metal ruthenium precursor and a solvent to obtain the mixed metal precursor solution;
optionally, the solvent comprises deionized water, ethanol, methanol, isopropanol, tetrahydrofuran, and other commonly used solvents;
optionally, the metal precursor is a metal salt;
optionally, the metal ruthenium precursor comprises ruthenium trichloride and/or ruthenium acetate;
optionally, the metal iron precursor comprises any one or a combination of at least two of ferric chloride, ferric nitrate or ferric sulfate;
optionally, the metal cobalt precursor comprises any one or a combination of at least two of cobalt chloride, cobalt nitrate, cobalt sulfate or cobalt acetate;
optionally, the metal nickel precursor comprises any one or a combination of at least two of nickel chloride, nickel nitrate or nickel sulfate;
optionally, the metal copper precursor comprises any one or a combination of at least two of copper chloride, copper nitrate or copper sulfate.

9. The preparation method according to claim 8, wherein the mixed metal precursor solution has a concentration of 0.001-0.2 g/mL;
optionally, the nitrogen-doped carbon suspension is obtained by mixing and dispersing nitrogen-doped carbon with a solvent;
optionally, the solvent comprises deionized water, ethanol, methanol, isopropanol or tetrahydrofuran;
optionally, the nitrogen-doped carbon suspension has a solid-liquid ratio of 1:(10-80) g/mL;
optionally, the dispersion is performed in a manner of ultrasonic dispersion for 0.5-12 h;
optionally, the impregnation is performed in a manner of stirring for 6-24 h;
optionally, the drying is performed at 80-120° C. for 6-12 h;
optionally, the reduction activation is performed in a hydrogen atmosphere;
optionally, the reduction activation is performed at 200-700° C. for 0.5-6 h.

10-12. (canceled)

13. A method for preparing an aromatic amino compound, comprising using the hydrogenation catalyst according to claim 1 in hydrogenation of an aromatic nitro compound.

14. The method according to claim 13, wherein the method comprises the following steps:
subjecting an aromatic nitro compound, as a raw material, and the hydrogenation catalyst, as a catalyst, to a reaction in a hydrogen atmosphere to obtain the aromatic amino compound.

15. The method according to claim 13, wherein the aromatic nitro compound comprises any one of compounds represented by Formula (VIII) to Formula (XVI): 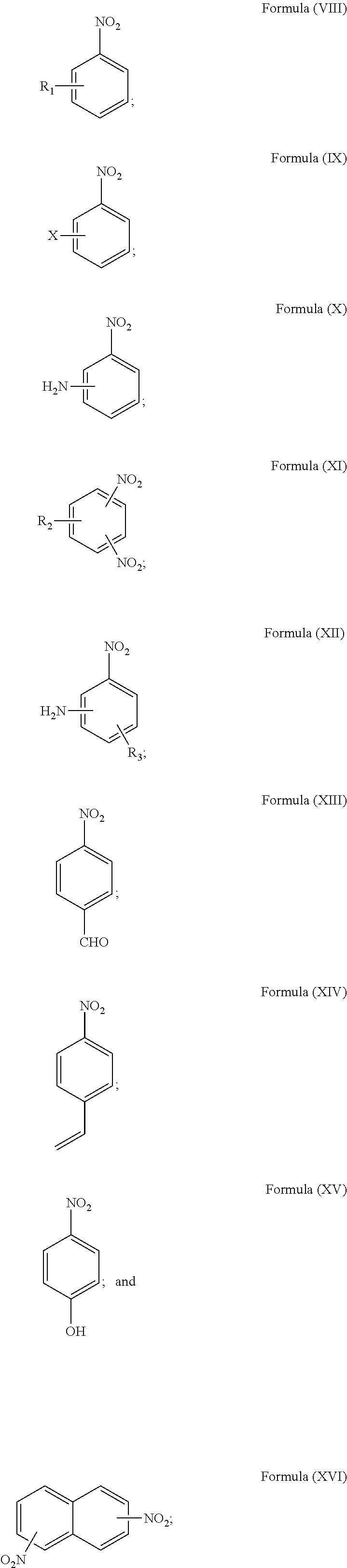
wherein R₁, R₂, and R₃ are independently selected from H or C1-C4 alkyl; X is selected from F, Cl or Br;
optionally, a usage amount of the catalyst is 0.1-30 wt. % by mass relative to the aromatic nitro compound;
optionally, the reaction is performed at −15° C. to 90° C. for 0.1-60 h with an initial pressure of 0.1-5 Mpa.
